(19) United States
(12) Patent Application Publication
(10) Pub. No.: US 2020/0075135 A1
(43) Pub. Date: Mar. 5, 2020

(54) TRIAL PLANNING SUPPORT APPARATUS, TRIAL PLANNING SUPPORT METHOD, AND STORAGE MEDIUM

(71) Applicant: Hitachi, Ltd., Tokyo (JP)

(72) Inventors: Hiroko OTAKI, Tokyo (JP); Kunihiko KIDO, Tokyo (JP); Haruhiko NISHIYAMA, Tokyo (JP)

(21) Appl. No.: 16/535,188

(22) Filed: Aug. 8, 2019

(30) Foreign Application Priority Data
Aug. 28, 2018 (JP) 2018-158954

(51) Int. Cl. G16H 10/20; G06F 16/28

(52) U.S. Cl. CPC G16H 10/20; G06F 16/285

(57) ABSTRACT

Provided is a trial planning support apparatus, including a processor and a storage unit, wherein the storage unit stores therein data of a plurality of documents about clinical trials implemented in the past, and wherein the processor is configured to receive information about a clinical trial, and search the plurality of documents for a plurality of sentences relevant to the received information, classify the plurality of sentences that have been found by the search into a plurality of clusters based on a degree of similarity, and output information about the sentences classified into clusters.

### CLAIM OF PRIORITY

The present application claims priority from Japanese patent application JP2018-158954 filed on Aug. 28, 2018, the content of which is hereby incorporated by reference into this application.

### BACKGROUND OF THE INVENTION

The present invention relates to a technology to support trial planning.

Japanese Patent Application Laid-open Publication No. 2011-159176 (Patent Document 1), for example, discloses a technology to support clinical trial planning. Patent Document 1 describes a feature map display method for clinical trial design that calculates and visually displays an index that characterizes the design based on two or more trial conditions that determine the design of a clinical trial (hereinafter referred to as a target clinical trial) targeting a prescribed disease, the method including a systematic clinical trial information extraction method for systematically extracting clinical trial information, an extraction information analysis method for analyzing the extracted clinical trial information, and know-how sharing method of information extraction/analysis methods.

### SUMMARY OF THE INVENTION

The process of new drug development starts from a basic study to select a potential compound from possible new drugs, followed by a non-clinical trial to study the medicinal pharmacological action using animals. Next, a clinical trial is conducted, and the results of the clinical trial are submitted to the Ministry of Health, Labor and Welfare to be reviewed. If the application is approved for manufacturing, the new drug can go to market.

A clinical trial is a trial conducted to examine the efficacy and safety of new drugs in humans. There is a need to secure trial data under high quality control, ensuring a sufficient number of subjects to demonstrate statistical significance with respect to efficacy and safety. Also, it is necessary to ensure trial data reliability and ethical consideration for the subjects. This makes clinical trials very costly, and if the clinical trial cannot be properly conducted and the development of a new drug fails, the pharmaceutical company would suffer huge losses. If a proper clinical trial cannot be conducted, it would cause the prolongation of the clinical trials and hence the delay in the release of new drugs, which would greatly affect patients.

In order to avoid such a situation, it is necessary for pharmaceutical companies to carefully consider the content of clinical trials and make plans, so that reliable trial data effective for new drug development is obtained. It is also necessary to prepare a trial execution plan (hereinafter referred to as a protocol) for implementation at medical facility such as a hospital, and to submit the protocol to the Ministry of Health, Labor and Welfare.

Information on the past clinical trials is very useful in formulating clinical trial plans, and it is common to formulate a plan with reference to similar clinical trials in the past. Furthermore, it is important to refer to clinical trial guidelines, treatment guidelines, and legal restrictions such as the Pharmaceutical Affairs Law in formulating a plan that allows the efficacy and safety to be examined in a scientific and ethical manner.

In order to effectively utilize the information of the past clinical trial plans, a database having stored therein clinical trial information is known. For example, public databases such as the PubMed service that can search abstracts of academic papers on the Internet and ClinicalTrials.gov, which is a website where protocols of clinical trials are registered, are available.

It is a common practice to comprehensively investigate protocols of a disease to be treated by the subject drug of a clinical trial and protocols of drugs having mechanisms similar to the subject drug, and formulate the trial conditions that match the purpose of the clinical trial.

In one trial method, the criteria for subject groups, i.e., who can participate in the trial, a method of implementing the trial, and the like are specified as the trial conditions. The method of implementing the trial includes study designs such as whether the trial requires a control group, whether the trial is blinded, whether the trial is randomized, whether the trial is joint efforts by multiple centers, and the doze and time interval at which the drug is to be administered, and items such as how to set the index (end point) to measure the efficacy, and events that are viewed as adverse events.

The setting of these trial conditions determines whether the sufficient results of the trial on the effectiveness and safety thereof can be obtained or not.

Criteria for the subject group include the selection criteria, which need to be met by the participants, and the exclusion criteria to define people who cannot participate in the trial based on factors such as age, sex, type and stage of disease, past medical history, and other medical conditions.

In the selection criteria and exclusion criteria, one sentence describes one factor, and those criteria are defined by multiple sentences. Below are the examples of the descriptions of the selection criteria and exclusion criteria.

### <Selection Criteria>

Patients from to 70 years old
Total bilirubin is less than 2.0 mg/dl
Platelet count is 70,000/mm3 or greater

### <Exclusion Criteria>

Patients who have been administered the same drug as the new drug in the past
Patients who have participated in another clinical trial during the past six months
Patients who are pregnant or breast-feeding

Patent Document 1 described above proposes the technology to analyze the relationship between the criteria for the subject group and clinical trials that have used those criteria.

The feature map display method of clinical trial design and display device described in Patent Document 1 define in advance the keywords characterizing the clinical trial conditions, create data that stores therein whether or not those keywords appeared in the sentences of the clinical trial conditions for each clinical trial, and then analyzes the relationship between the clinical trial condition data and the clinical trial groups categorized by disease and the like. Thus, it is necessary to include and structure the keywords that characterize the trial conditions, on the premises that the features of the trial conditions to be analyzed are known in advance.

However, the items set for the trial conditions for a clinical trial greatly vary and are very complex, and because there are no predetermined templates, the conditions are written in free text. For this reason, it is not easy to study, analyze and organize the keywords that characterize the conditions that vary in descriptions (problem of descriptive variations).

Also, the decisions on which information needs to be studied and analyzed in what way in terms of the trial conditions largely depend on the know-how of the experienced trial administrators. Even a person with experiences sometimes have difficulties to properly decide a point of view of the analysis since rules for clinical trials are complex, some clinical trials are implemented as a large project, some clinical trials require multiple points of view, and some clinical trials continue to evolve (problem regarding data aggregation).

There is also a relationship between conditions where two conditions are set in association with each other, and it is a common practice that when one trial condition is set, all possible associated conditions are listed. When an adverse event could happen in a kidney, for example, patients with kidney failure are to be excluded. Although it was desirable to analyze the settings of past clinical trials, analyze the relationship between the conditions, and analyze conditions that are more likely to be set for a certain condition, information about the clinical trial design greatly varies, and does not have specific templates, which made it difficult to analyze the relationship between conditions (problem in analyzing clinical trial conditions).

The clinical trial design such as criteria for a subject group and a method of implementing a trial in the trial conditions greatly affects the results of the clinical trial, but a technology to properly analyze clinical trial information and to support the clinical trial planning has not been disclosed.

The present invention was made to solve the problems described above, and an object thereof is to provide a trial planning support apparatus that properly classifies trial conditions of clinical trials targeting a certain disease or action mechanism, extracts features of the trial conditions included in the classification, and visually displays the trial conditions using diagrams, a trial planning support method and a trial planning support program. For the respective trial conditions, information regarding the association with trial result information will also be displayed.

In order to solve at least one of the foregoing problems, provided is a trial planning support apparatus, comprising: a processor; and a storage unit, wherein the storage unit stores therein data of a plurality of documents about clinical trials implemented in the past, and wherein the processor is configured to: receive information about a clinical trial, and search the plurality of documents for a plurality of sentences relevant to the received information; classify the plurality of sentences that have been found by the search into a plurality of clusters based on a degree of similarity; and output information about the sentences classified into clusters.

According to one embodiment of the present invention, it is possible to comprehensively analyze information in designing and evaluating a clinical trial by converging descriptions with very small differences and classifying those descriptions so that clinical trial information is properly extracted and analyzed.

Challenges, configurations, and effects other than those described above will become apparent in the descriptions of embodiments below.

### BRIEF DESCRIPTION OF THE DRAWINGS

FIGS. 1A and 1B are block diagrams illustrating a functional configuration of a trial planning support apparatus of an embodiment of the present invention.

FIG. 2 is a flowchart for explaining a word vector representation collection process performed by the trial planning support apparatus of the embodiment of the present invention.

FIG. 3 is a flowchart for explaining a sentence vector representation collection process performed by the trial planning support apparatus of the embodiment of the present invention.

FIG. 4 is a flowchart for explaining a sentence vector representation clustering process performed by the trial planning support apparatus of the embodiment of the present invention.

FIG. 5 is a flowchart for explaining a display process of data that has undergone clustering by the trial planning support apparatus of the embodiment of the present invention.

FIG. 6 is a diagram explaining a data example of condition sentences of clinical trials stored in the trial planning support apparatus of the embodiment of the present invention.

FIGS. 7A to 7C are diagrams explaining a data example of a parameter value extraction result generated by the trial planning support apparatus of the embodiment of the present invention.

FIGS. 8A and 8B are diagrams explaining a data example of a sentence cluster generated by the trial planning support apparatus of the embodiment of the present invention.

FIG. 9 is a diagram explaining a display example of a screen to enter a disease name, action mechanism name, and drug name by the trial planning support apparatus of the embodiment of the present invention.

FIG. 10 is a diagram explaining a display example of a trial planning support screen by the trial planning support apparatus of the embodiment of the present invention.

FIG. 11 is a flowchart for explaining a clinical trial condition classification process performed by the trial planning support apparatus of the embodiment of the present invention.

FIG. 12 is a flowchart for explaining a sentence phrase division and a phrase cluster determining process performed by the trial planning support apparatus of the embodiment of the present invention.

FIG. 13 is a block diagram illustrating a sentence division performed by the trial planning support apparatus of the embodiment of the present invention.

FIG. 14 illustrates an example of a modification structure between words that is referred to by the trial planning support apparatus of the embodiment of the present invention.

FIG. 15 illustrates an example of a semantic structure that is referred to be the trial planning support apparatus of the embodiment of the present invention.

FIG. 16 is a diagram illustrating an example of relationship data between clinical trials and sentence clusters generated by the trial planning support apparatus of the embodiment of the present invention.

### DETAILED DESCRIPTION OF THE EMBODIMENTS

The trial planning support method implemented by a trial planning support apparatus of one embodiment of the present invention is constituted of a clinical trial information classification method, a clinical trial information analysis method, and clinical trial information relationship analysis method.

The clinical trial information classification method is a method of classification based on the similarity of the description of the trial conditions described in free text. This method is used for classifying the criteria for subject groups, i.e., who can participate in the trial, or trial methods that define a method of implementing the trial, and the like.

In order to classify the descriptions in free text, the following configuration is employed.

The clinical trial information classification method of one embodiment of the present invention, for example, includes a document acquisition and collection unit that obtains a document to be analyzed by narrowing down documents based on disease, drug, action mechanism, and the like; a word vector representation collection unit that uses the obtained document to represent words in vectors; a sentence vector representation collection unit that uses word vectors to represent sentences in vectors; and a sentence clustering unit that classifies sentences using the sentence vectors.

One of the features of the clinical trial information classification method of this embodiment is to divide clinical trial information into meaningful units such as sentences or phrases, convert the sentences or phrases into vectors, and classify the sentences based on the degree of similarity of the meaning of the sentences. In the information about the clinical trial design such as trial conditions, what kind of index is to be set, and what kind of value is to be set for that index are important, and even if there are variations in descriptions of the information about the trial design, which are described in various manners, similar indices need to be classified into the same group. Therefore, classifying indices based on the similarity to each other is also one of the features of the clinical trial information classification method of this embodiment.

The clinical trial information analysis method is a method of analyzing the trial condition group classified by the clinical trial information classification method, and analyzing and presenting words and values characterizing a cluster. To present features of classified sentence clusters, the following configuration is employed.

For example, the clinical trial information analysis method of one embodiment of the present invention is constituted of a trial parameter value extraction unit that extracts important indices and statistical values for a clinical trial from sentences. The clinical trial information analysis method is characterized by the fact that indices that characterize the classified clinical trial information and values set for those indices are extracted and undergo a statistical analysis, and the distribution of the values is visualized.

The trial condition classification relationship analysis unit analyzes whether or not there is a condition that is always set when a certain condition is set, and refers to the relevance in the past cases when formulating a protocol, so that relevant conditions are presented.

Therefore, the trial condition classification relationship analysis unit includes a co-occurring relationship data creation unit that creates co-occurring relationship data between the clinical trial conditions set in one clinical trial, and a clinical trial condition presentation unit that presents relevant clinical trial conditions in the process of setting clinical trial conditions. The analysis results on the relevance can also be used for data to calculate the presentation order when presenting the classification.

Below, a trial planning support apparatus in a preferred embodiment of the present invention will be explained in detail with reference to figures.

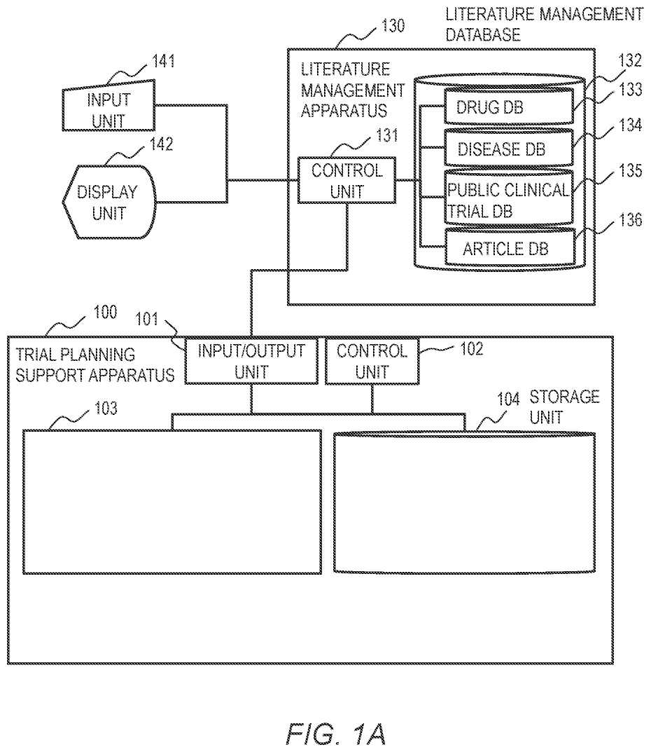

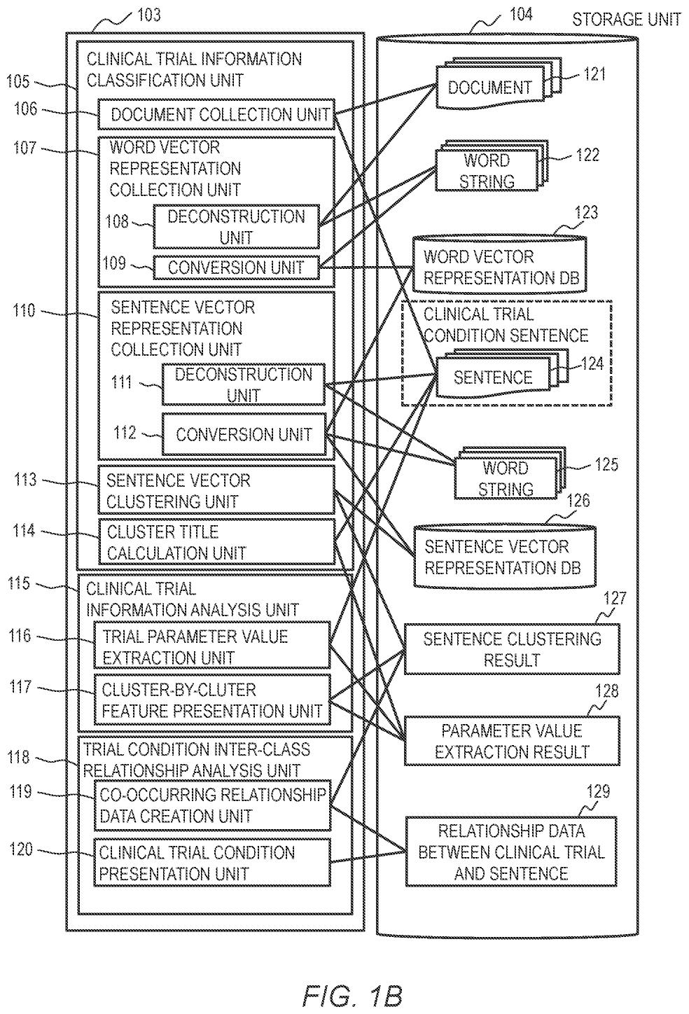

FIGS. 1A and 1B are block diagrams illustrating a functional configuration of a trial planning support apparatus 100 of an embodiment of the present invention.

This trial planning support apparatus 100 is an apparatus that supports formulation of a clinical trial execution plan (protocol). As illustrated in FIG. 1A, the trial planning support apparatus includes an input/output unit 101, a control unit 102, a memory 103, and a storage unit 104.

The input/output unit 101 is an interface that exchanges data between the trial planning support apparatus 100 and another device connected to the trial planning support apparatus 100 (literature management apparatus 130 in the example of FIG. 1A).

The control unit 102 is a processor that performs various processes in accordance with programs stored in the memory 103. The memory 103 is a storage device that stores therein the programs executed by the control unit 102 and data referred to by the control unit 102, and the like. In the example of FIG. 1B, the memory 103 stores therein a clinical trial information classification unit 10 105, a clinical trial information analysis unit 115, and a trial condition classification inter-class analysis unit 118. In actuality, those units are realized by the programs stored in the memory 103. That is, the processes performed by those units in the descriptions below are actually executed by the control unit 102 in accordance with the programs stored in the memory 103.

A storage unit 132 in the literature management apparatus 130 stores therein literature data about clinical trials (that is, clinical trials of treatment) such as a drug database 133 having stored therein the names of drugs developed in the past and drug pharmacological actions, a disease database 134 having stored therein the names of diseases, and an article database 136 having stored therein articles describing clinical trials implemented in the past, and a public clinical trial database 135 (will be collectively referred to as literature management database). Although these databases are stored in the storage unit 132 of the literature management apparatus 130 in the example of FIG. 1A, those databases may be stored in the storage unit 104, or at least some of the databases may be stored in the storage unit 104 as necessary.

The literature data is associated with drug data and disease data so that documents can be filtered based on drugs and action mechanisms, or the diseases.

The storage unit 104 of the trial planning support apparatus 100 stores therein a document 121, a word string 122, a word vector representation database 123, a clinical trial condition sentence 124, a word string 125, a sentence vector representation database 126, a sentence clustering result 127, a parameter value extraction result 128, and relationship data between clinical trial and sentence 129.

The clinical trial information classification unit 105 includes a document collection unit 106, a word vector representation collection unit 107, a sentence vector representation collection unit 110, a sentence vector clustering unit 113, and a cluster title calculation unit 114.

The document collection unit 106 collects data of the public clinical trial database 135 and data of the article database 136 associated with each other based on the disease, drug, action mechanism of the drug, and the like. When information regarding a clinical trial such as a disease, drug or action mechanism of the drug is provided, for example, the document collection unit 106 looks up sentences relevant to that information in the drug database 133, the disease database 134, the public clinical trial database 135, and the article database 136 or the like, and stores the retrieved sentences in the storage unit 104 as the document 121. Specifically, when a trial of a diabetes treatment drug is to be implemented, for example, a sentence relevant to diabetes may be searched, or a document relevant to drugs similar to the treatment drug may be searched.

The word vector representation collection unit 107 is a processing unit that converts a word into a vector representation using a set of documents 121 collected and accumulated by the document collection unit 106 and that stores the word in the vector representation database 123, and includes a deconstruction unit 108 and a conversion unit 109.

The deconstruction unit 108 reads a document 121 from the storage unit 104, divides that document into structural units by detecting a space or through morphological analysis, and generates a word string 122. As a result, a word string 122 for each document is stored in the storage unit 104. If the document 121 is in English, the deconstruction unit 108 may divide the document 121 at each space. If the document 121 is in Japanese, the deconstruction unit 108 may divide the document 121 by the morphological analysis.

The conversion unit 109 converts each of the word strings obtained by the deconstruction unit 108 into a vector string with reference to the word vector representation database 123. The conversion unit 109 may convert the word strings into the appearance frequency, appearance position, and the like as the vector representation, for example. LSI (Latent Semantic Indexing), tfidf, or the like may be used for the conversion to the appearance frequency. The word2vec or the like may be used for the conversion to the appearance position. The vector representation is represented by a vector string. In this way, a vector representing each word is generated such that vectors representing words that are more likely to co-occur have values closer to each other.

The sentence vector representation collection unit 110 is a processing unit that converts a clinical trial condition sentence into a word string 125, converts the word string 125 to a sentence vector representation using the word vector representation, and stores the sentence vector representation in the sentence vector representation database 126. The sentence vector representation collection unit 110 includes a deconstruction unit 111 and a conversion unit 112.

The deconstruction unit 111 reads a document 121 from the storage unit 104, divides that document into structural units by detecting a space or through morphological analysis, and generates a word string 125, in a manner similar to the deconstruction unit 108 of the word vector representation collection unit 107.

The conversion unit 112 converts respective words of a sentence, which were converted to the word string 125, into the vector representation stored in the vector representation database 123, and obtains sentence vector representation by averaging the vector representation of the word string 125 constituting a sentence, for example.

The sentence vector clustering unit 113 clusters the sentence vectors stored in the sentence vector representation database 126 based on a degree of similarity (more precisely, based on the degree of similarity of vectors that represent those. Clustering may be performed by hierarchical clustering or other clustering methods such as K-means method. The clustering result is stored as the sentence clustering result 127.

The clinical trial information analysis unit 115 includes a trial parameter value extraction unit 116 and a cluster-by-cluster feature presentation unit 117. The trial parameter value extraction unit 116 is a processing unit that extracts, from each trial condition sentence, indices considered important as trial conditions such as indices relevant to clinical examinations, names relevant to drugs, and names relevant to various treatments, as well as numerical values relevant to the indices, and that stores those indices and values in the parameter value extraction result 128.

The text string numbers indicating respective indices and values may be stored in the parameter value extraction result 128 so that the corresponding relationship with the original sentence is saved.

The cluster-by-cluster feature presentation unit 117 obtains the relevant trial parameter value extraction result data for each cluster from the sentence clustering result 127. Specifically, the cluster-by-cluster feature presentation unit 117 is a processing unit that analyzes an index appearing in each cluster and relevant values based on the appearance frequency, and present the features thereof.

The trial condition classification inter-class analysis unit 118 includes a co-occurring relationship data creation unit 119 and a clinical trial condition presentation unit 120. The co-occurring relationship data creation unit 119 is a processing unit that totals up the sentence clustering result 127 for each trial, creates binary relationship data of clusters co-occurring in the trial, and stores the data as the relationship data between clinical trial and sentence 129.

Once one cluster is specified, the other cluster can be presented by referring to the relationship data between clinical trial and sentence 129.

Next, the process flow will be explained. First, the procedures of the vector representation collection process by the word vector representation collection unit 107 will be explained.

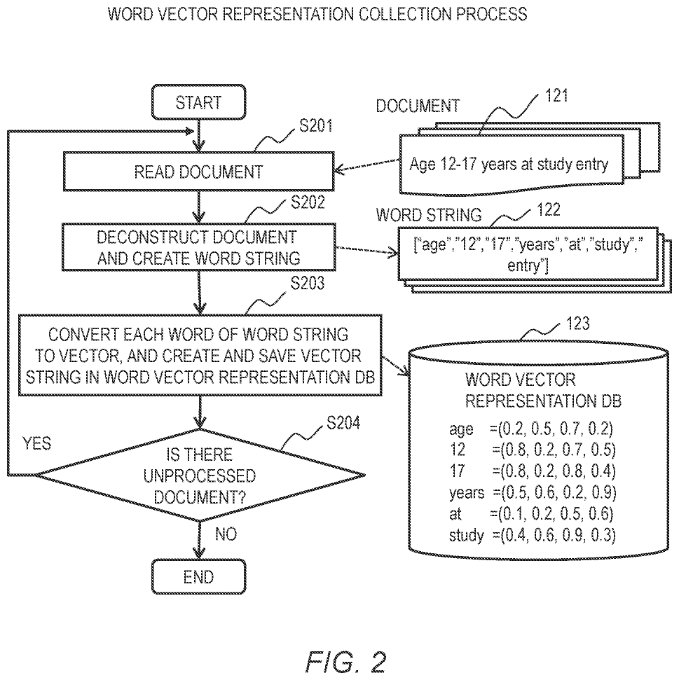

FIG. 2 is a flowchart for explaining the word vector representation collection process performed by the trial planning support apparatus 100 of the embodiment of the present invention.

As illustrated in FIG. 2, in the word vector representation collection unit 107, the deconstruction unit 108 reads one document 121 from the set of documents 121 in Step S201, and deconstructs the document 121 into constituting units, thereby creating a word string in Step 202. Using “Age 12-17 years at study entry.” as an example, because this document is in English, the deconstruction unit 108 deconstructs the sentence at a space and symbol, and as a result, the word string 122 is represented as follows: “age,” “12,” “17,” “at,” “study,” “entry.” This word string 122 is stored in the storage unit 104.

After the word string 122 is created, the conversion unit 109 converts each word in the string into a vector, creates a vector string, and stores the vector string in the word vector representation database 123 in Step S203. In the word vector representation database 123, a vector of (0.2, 0.5, 0.7, 0.2) is stored for the word “age,” and a vector (0.8, 0.2, 0.7, 0.5) is stored for the word “12.”

In Step S204, the word vector representation collection unit 107 determines whether or not there is a document 121 that has not yet been processed in the group of documents 121. If there is an unprocessed document 121, the word vector representation collection unit 107 returns to Step S201, and repeats the steps described above. If there is no unprocessed document 121, the word vector representation collection unit 107 ends the process.

The constituting unit for deconstructing the document 121 may be a letter, or a series of letters (N-gram).

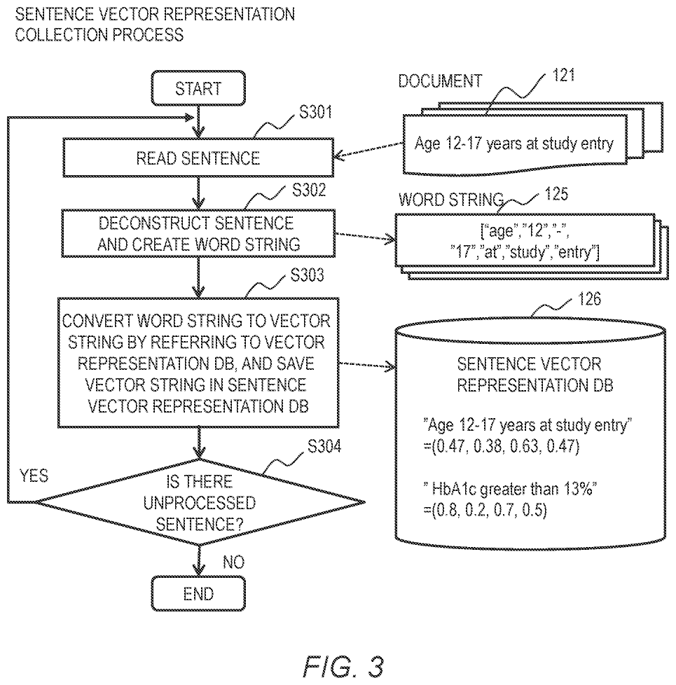

FIG. 3 is a flowchart for explaining a sentence vector representation collection process performed by the trial planning support apparatus 100 of the embodiment of the present invention.

In FIG. 3, in the sentence vector representation collection unit 110, a deconstruction unit 111 reads a sentence, and deconstructs the sentence into strings of words in Steps S301 and S302. Next, the conversion unit 112 converts the strings of words into vector strings with reference to the word vector representation database 123, and stores the vector strings in the sentence vector representation database 126 in Step S303.

For example, the conversion unit 12 converts the respective words “age,” “12,” “17,” “years,” “at,” “study,” and “entry” into respective word vectors, and adds up and averages those vector values to obtain a vector of the sentence “Age 12-17 years at study entry.”

In Step S304, the sentence vector representation collection unit 110 determines whether or not there is a document 121 that has not yet been processed in the group of documents 121. If there is an unprocessed document 121, the sentence vector representation collection unit 110 returns to Step S301, and repeats the steps described above. If there is no unprocessed document 121, the sentence vector representation collection unit 110 ends the process.

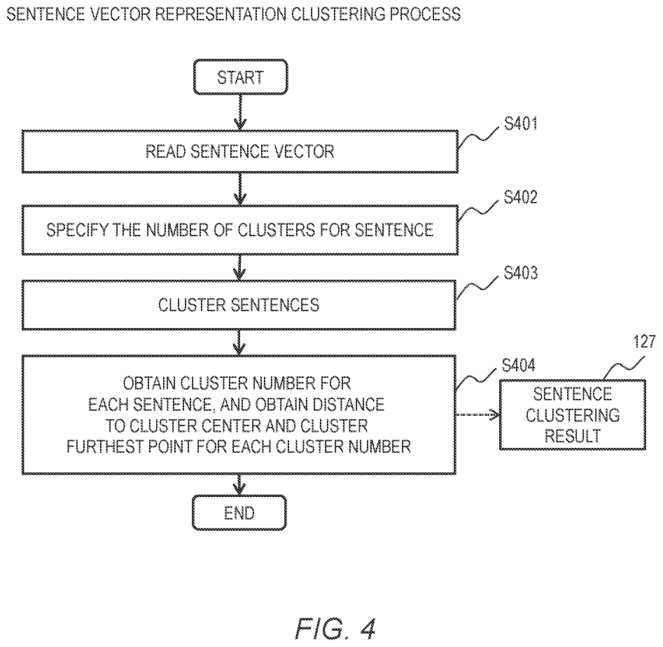

FIG. 4 is a flowchart for explaining a sentence vector representation clustering process performed by the trial planning support apparatus 100 of the embodiment of the present invention.

In FIG. 4, the sentence vector clustering unit 113 reads the sentence vector with reference to the sentence vector representation database 126 in Step S401. The sentence vector clustering unit 113 specifies the number of clusters in Step S402, and clusters the sentence in Step S403.

In Step S404, the sentence vector clustering unit 113 stores a cluster number in each sentence that went through clustering. The sentence vector clustering unit 113 further stores therein the distance of the center and the furthest point of each cluster. The K-means method, the hierarchal clustering method, and the like may be used for the clustering method.

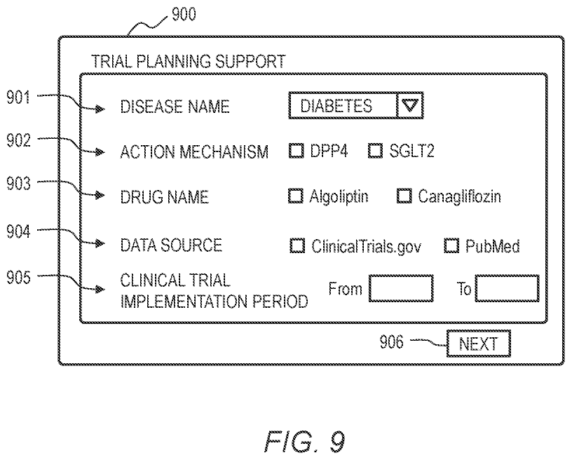

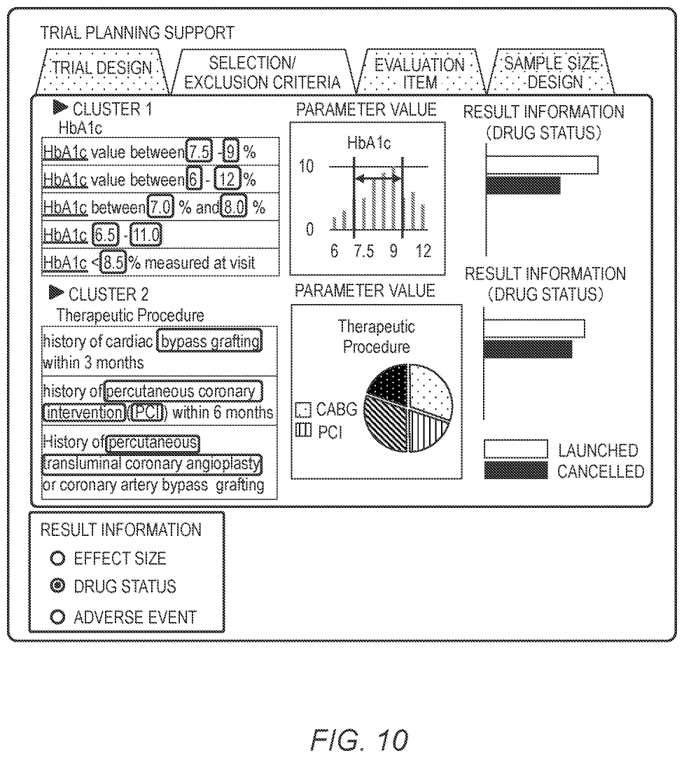

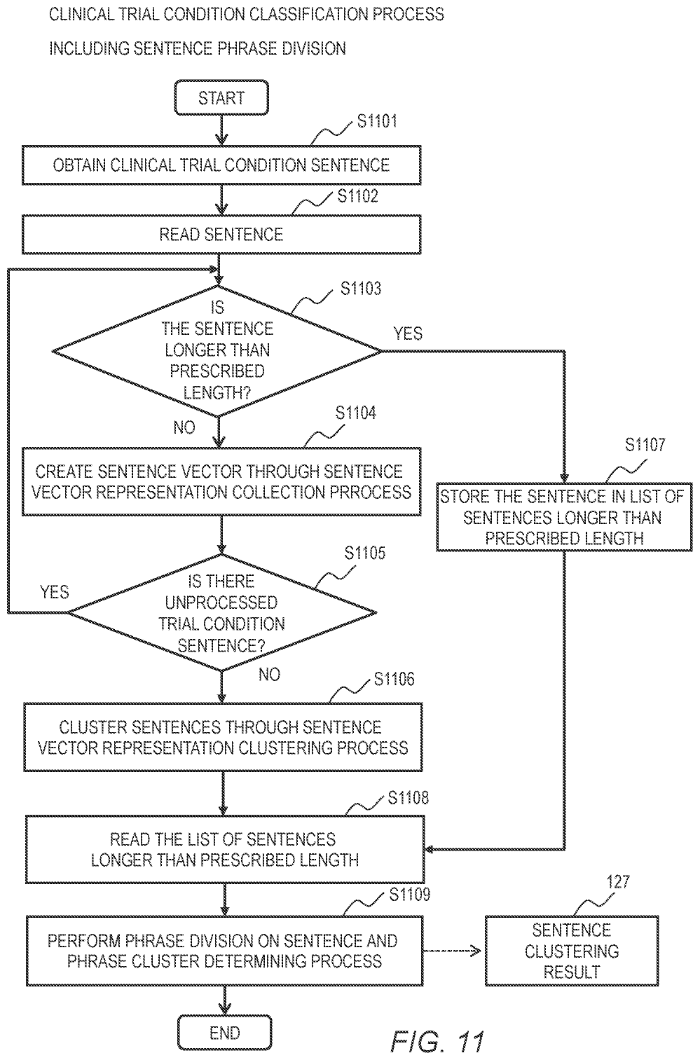

FIG. 9 is a diagram explaining a display example of a screen to enter a disease name, action mechanism name, and drug name by the trial planning support apparatus 100 of the embodiment of the present invention.

A display screen 900 illustrated in FIG. 9 displays a disease name pull-down menu 901 for selecting a disease name from a pull-down menu, and an action mechanism check box 902 that displays action mechanisms associated with the disease name for selecting an action mechanism by checking the box, and a drug name check box 903 that displays drug names associated with the disease name for selecting a drug name by checking the box. The display screen 900 may alternatively have input fields through which a disease name, action mechanism, and drug name can be searched by inputting texts, instead of the pull-down menu or check box described above.

The display screen 900 further includes a data source check box 904 for selecting a data source for referring to the information of the existing clinical trial plans, and input boxes 905 for entering the start time and end time of the period of a target clinical trial to specify the implementation time period of the target clinical trial.

After a disease name to be tested in a clinical trial is selected from the disease name pull-down menu 901, an action mechanism name to be tested in a clinical trial is selected from the action mechanism check box 902, a drug name to be tested in a clinical trial is selected from the drug name check box 903, and the next screen button is pressed, the control unit 102 starts a process to display the clustered data.

The screen illustrated in FIG. 9 may be displayed by the control unit 102 generating and outputting data for displaying the screen, and the display unit 142 performing display based on the data. The same applies to the screen of FIG. 10 described below and the like.

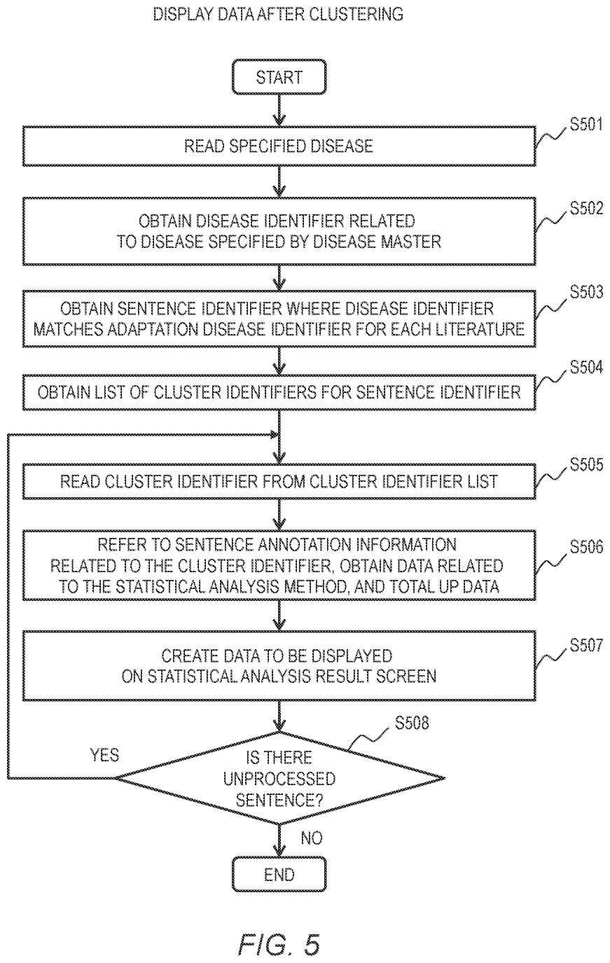

FIG. 5 is a flowchart for explaining a display process of data that has undergone clustering by the trial planning support apparatus 100 of the embodiment of the present invention.

When a disease name, action mechanism name, and drug name are selected as described above, the control unit 102 reads the selected disease name, action mechanism, and drug name (Step S501). Then, the control unit 102 refers to the literature management database in the literature management apparatus 130 to search for the disease master, the action mechanism master, and the drug master, and obtains identifiers of the disease, the action mechanism and the drug relevant to the disease name, action mechanism name and drug name (Step S502). Although FIG. 5 is a flowchart for explaining the example of the disease name, the same process is performed for the drug and action mechanism.

Next, the control unit 102 refers to the literature management database in the literature management apparatus 130, searches for the adaptation disease data by document, and obtains the identifier of sentences associated with the disease that was entered through the input unit based on the adaptation disease identifier (Step S503). Next, the control unit 102 searches for sentence cluster data, and obtains an identifier of a cluster corresponding to the sentence identifier (Step S504).

The control unit 102 then reads the identifier of the cluster (Step S505), refers to the sentence annotation information about the identifier, counts the number of times the disease name, drug name, treatment name, clinical trial name and the like are used in the sentence, and totals up for each trial (Step S506).

Next, the control unit 102 creates data to be displayed in the statistical analysis result screen (FIG. 10).

Next, the control unit 102 determines whether there is an unprocessed cluster in the set of clusters or not (Step S508). If there is an unprocessed cluster, the process returns to Step S505, and repeats the steps described above for the unprocessed cluster. If there is no unprocessed cluster, the process is ended, and the trial planning support screen is displayed through the input/output unit 101.

FIG. 10 is a diagram explaining a display example of the trial planning support screen by the trial planning support apparatus 100 of the embodiment of the present invention.

FIG. 10 illustrates a display example of the information about the selection criteria and exclusion criteria. In the display example, information about at least one (two, for example) cluster of the plurality of clusters obtained by clustering is displayed. For example, for one cluster (Cluster 1 of FIG. 10), the sentences classified into the cluster (“HbA1c value between 7.5-9%” for example) and the text string charactering that cluster (“HbA1c” for example) are displayed.

In the screen, information representing the index and values thereof included in each sentence may also be displayed. In the example of “HbA1c value between 7.5-9%” of FIG. 10, “HbA1c” is the index, and “7.5” and “9” are the values corresponding to the index, which are highlighted.

If a plurality of sentences classified into a cluster respectively include different values relevant to the same index, the appearance frequency distribution of those values may also be displayed. In the example of FIG. 10, a histogram of the number of occurrences of HbA1c values (such as “7.5%” and “9%”) is displayed as the “parameter value”. If such values have a guideline such as “7.0%-9.5%,” for example, the range of the guideline may also be displayed.

For another cluster (Cluster 2 of FIG. 10) as well, the sentences classified into the cluster (“history of cardiac bypass grafting within 3 months”) and the text string charactering that cluster (“Therapeutic Procedure”) are displayed. In a manner similar to the example of Cluster 1, information representing the index and values thereof included in each sentence may also be displayed.

In the example of “history of cardiac bypass grafting within 3 months” of FIG. 10, since “bypass grafting” is the index, this section is highlighted. If a plurality of sentences classified into a cluster respectively include different indices, specifics of those may also be displayed as the “parameter values”. In the example of FIG. 10, the number of occurrences of each index is displayed as a pie chart.

Furthermore, in the example of FIG. 10, the statistics of the results about the condition sentences are also displayed, based on the information such as whether the drug for which the trial condition sentence in each cluster was used actually went to market or the trial was cancelled halfway, and the like. Specifically, for the trial condition sentences in the respective clusters, the number of condition sentences for the drugs that went to market (launched), and the number of condition sentences for the drugs that ended up with cancellation of the trial (cancelled) may also be displayed.

Information about the design of clinical trials is very complex, and written in free text without predetermined templates. With this embodiment, however, the design related information of clinical trial can be classified based on a degree of similarity of meanings of words, and each classification unit may be subjected to analysis. The trial planning support screen of FIG. 10 also displays information regarding indices and values thereof set for the past clinical trials in the designing stage, which makes it possible to refer to those setting values.

Furthermore, it is possible to see the results of the past clinical trials of certain design. In the example of FIG. 10, the results were displayed to show whether the drugs relevant to the sentences in each cluster went to market or not, but it is also possible to display information that affects the results of clinical trials such as the size of the end point of the drug (effect size), the appearance probability of adverse events, and the time required to find test subjects.

In the example of FIG. 10, the trial result for each classification is displayed, but because the trial result changes depending on the size of the index value, the user may specify the scope of the values in a graph indicating the index values, and the trial result may be displayed with the specified scope.

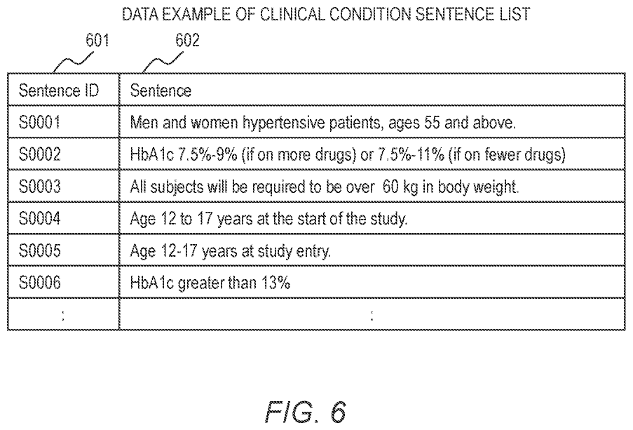

FIG. 6 is a diagram explaining a data example of the condition sentences of clinical trials stored in the trial planning support apparatus 100 of the embodiment of the present invention.

Each sentence is assigned with an identifier 601, and includes sentence information 602. The sentence saved as the sentence information 602 corresponds to the clinical trial condition sentence 124 of FIG. 1B. The table of FIG. 6 may also include information associating a sentence in the sentence information 602 with the document from which that sentence was extracted (such as an identifier of the document). The sentence or the document itself is associated with the disease name, drug name, and action mechanism name.

The clinical trial condition sentence in the sentence information 602 may be a string of words indicating the trial condition such as “HbA1c greater than 13%,” and does not have to meet the grammatical requirements such as having to include a subject and object. The same applied to the sentences handled by the sentence vector clustering unit 113.

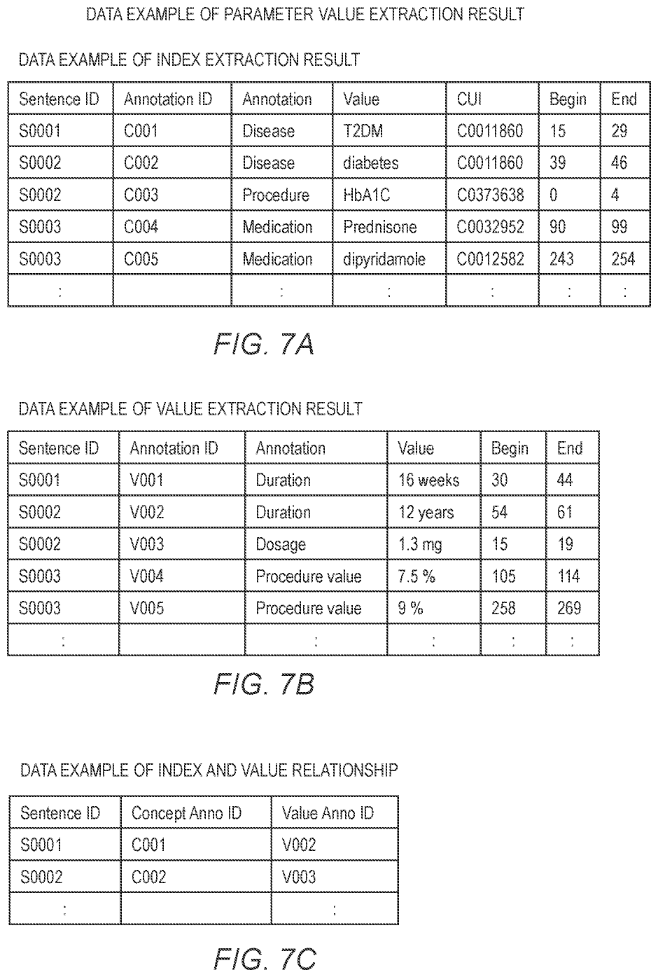

FIG. 7A to 7C are diagrams explaining a data example of the parameter value extraction result 128 generated by the trial planning support apparatus 100 of the embodiment of the present invention.

Specifically, FIG. 7A to 7C illustrate a data example of the parameter extraction result 128 after the trial parameter value extraction unit 116 extracts, from each trial condition sentence 124 of FIG. 6, indices considered important as trial conditions such as indices relevant to clinical examinations, names relevant to drugs, and names relevant to various treatments, as well as numerical values relevant to each index. FIG. 7A is a data example as a result of extracting the indices, and FIG. 7B is a data example of extracting values relevant to each index.

The parameter value extraction result 128 includes a sentence identifier (sentence ID), index extracted from sentences (information indicating what the index is relevant to) or an identifier for index given to each value of the index (Annotation ID), a value indicating the category of the index to show the type of index of the extraction result (Annotation), text string of extracted indices or values of the indices (Value), and the start point (Begin) and end point (End) of the text string indicating the index names or values.

The start point (Begin) and end point (End) may be numerical values indicating the positions of the first letter and last letter of the text string in the sentence. As a result, the corresponding relationship between the original sentence and the text string of the indices extracted therefrom is saved.

The data relevant to the index of FIG. 7A may further include an identifier (CUI) of a dictionary used to extract the indices.

The parameter value extraction result 128 may further include relationship data between each index and values as illustrated in FIG. 7C. This data includes, for example, information that associates the sentence identifier (Sentence ID), the identifier of each index extracted from the sentence (Concept Anno ID), and the identifier of the value of the index (Value Anno ID) with each other.

When the clinical trial condition sentence is “HbA1c greater than 13%,” for example, the trial parameter value extraction unit 116 may extract and register “HbA1c” as the text string (value) of index in FIG. 7A, and extract and register “13%” as the text string (value) of the value of the index in FIG. 7B. In this case, “HbA1c” and “13%” are associated with each other by the data of FIG. 7C.

That is, the text string of “index” extracted here represents the concept of the index with which the value is associated. Alternatively, the “index” (such as “HbA1c”) and the “value” (such as “13%”) related thereto may also be referred to as “parameter attribute” and “parameter value.” Examples of the index include a disease name, a clinical trial name, a drug name, an action mechanism name, and a treatment name.

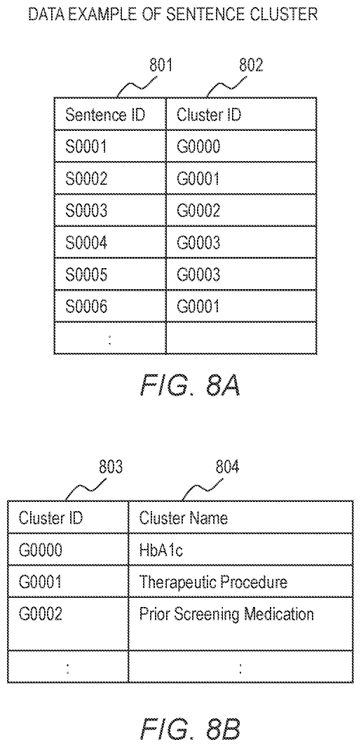

FIGS. 8A and 8B are diagrams explaining a data example of the sentence cluster generated by the trial planning support apparatus 100 of the embodiment of the present invention.

The data shown in FIG. 8A includes an identifier (Sentence ID) 801 of each sentence and an identifier (Cluster ID) 802 of a cluster to which each sentence belongs. This data is obtained as a result of conducting the sentence vector representation clustering process (FIG. 4), and is included in the sentence clustering result 127.

The cluster title calculation unit 114 calculates a title representing the content of the cluster, and saves the calculated title for each cluster. This title is displayed in the trial planning support screen as illustrated in FIG. 10. FIG. 8B shows an example of the cluster title data. The example of FIG. 8B includes an identifier of each cluster (Cluster ID) 803 and the title of each cluster (Cluster Name) 804.

The cluster title calculation unit 114 may extract a feature word using the TF-IDF method or the like, for example, from the sentences in the cluster and the entire data subjected to clustering, and use the feature word as the title of the cluster. Alternatively, a sentence in the cluster including the word obtained as the feature word may be used for the title.

In some cases, the trial condition sentences include a sentence made up of a plurality of trial conditions. If one sentence includes a plurality of conditions, the trial planning support apparatus 100 may perform the clinical trial condition classification process in which one sentence is divided into a plurality of phrases (sections of sentence) such that one condition is included in one phrase, and the obtained phrases are classified to clusters of the condition sentences.

FIG. 11 is a flowchart for explaining the clinical trial condition classification process performed by the trial planning support apparatus 100 of the embodiment of the present invention.

First, the control unit 102 obtains the clinical condition sentences 124 in Step S1101, and reads one clinical condition sentence in Step S1102. The control unit 102 determines whether the read clinical condition sentence is longer than a prescribed length or not in Step S1103.

If the sentence does not exceed the prescribed length, the sentence vector representation collection unit 110 creates sentence vectors in Step S1104. The control unit 102 determines whether there is an unprocessed sentence among the obtained clinical trial condition sentences 124, and if so, the control unit 102 performs Steps S1102 to S1104 on an unprocessed sentence.

Next, in Step S1106, the sentence vector clustering unit 113 clusters sentences using the sentence vectors created in Step S1104. On the other hand, if the control unit 102 determines that the clinical trial condition sentence 124 is longer than the prescribed length in Step S1103, the condition sentence is stored in the list of sentences longer than the prescribed length in Step S1107.

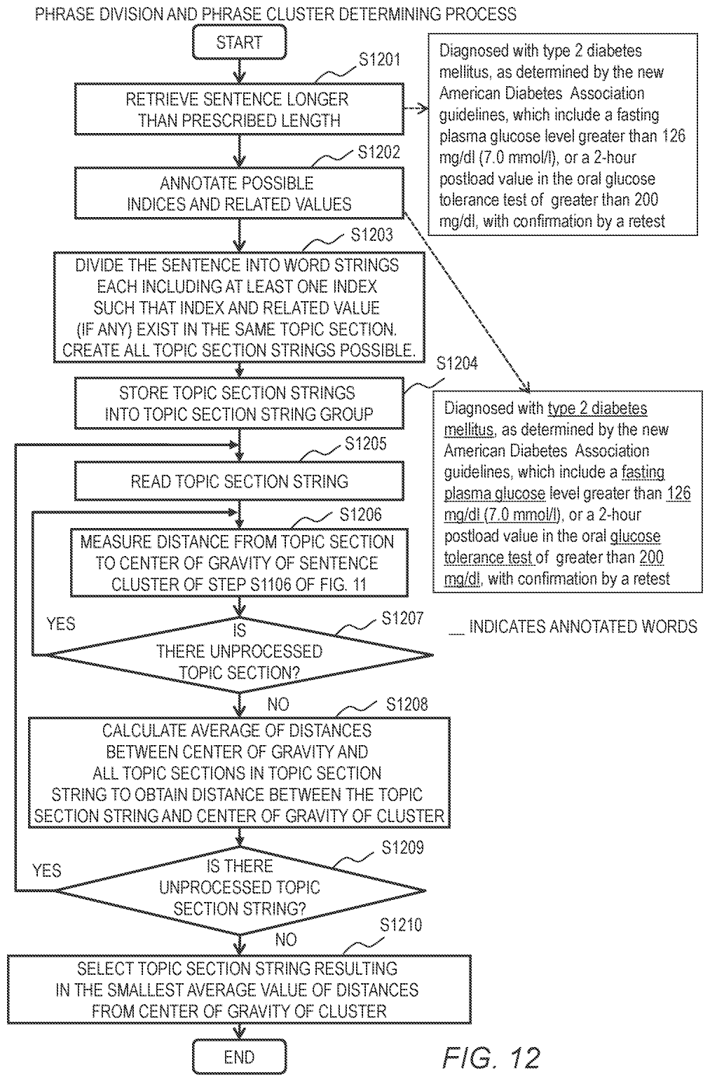

The control unit 102 reads the list of sentences longer than the prescribed length in Step S1108, performs the phrase division and phrase cluster determining process on sentences in Step S1109, and stores the clustering result of the phrases in the sentence clustering result. The process of Step S1109 will be explained in detail with reference to FIG. 12.

FIG. 12 is a flowchart for explaining the sentence phrase division and the phrase cluster determining process performed by the trial planning support apparatus 100 of the embodiment of the present invention.

The clinical trial condition sentence 124 is characterized by the fact that the index charactering the condition, and the value of the index and the unit of the value appear in the same sentence. Examples of the index include a disease name, a clinical trial name, and a drug name. Examples of the value include a clinical trial value and a dose, which are relevant to the index.

In some cases, the clinical trial condition sentence 124 is described such that a plurality of conditions are combined into one sentence, and generally, it is preferable that such a sentence be divided by condition, and classified into a cluster corresponding to each condition. In order to realize that, a process to divide a sentence by condition is necessary. The process of FIG. 12 is to divide one sentence made up of a plurality of conditions, and to determine the cluster for each condition.

In Step S1201, the control unit 102 retrieves a sentence longer than a prescribed length. This is one of the sentences stored in the list in Step S1107 of FIG. 11. In Step S1202, the control unit 102 annotates possible indices and relevant values. Examples of the index include a disease name, a clinical trial name, and a drug name. Example of the values of index include a clinical trial value and a dose. The annotation method may be a method using a dictionary or regular expression or a method using machine learning.

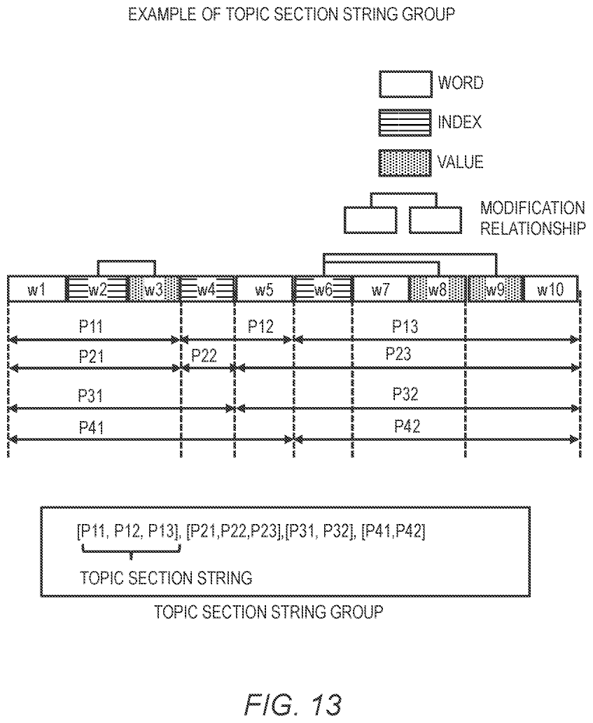

In Step S1203, the control unit 102 divides the target sentence into a plurality of text strings such that each text string includes at least one index. The text string obtained by the division corresponds to the phrase in the description above (or a section of the sentence), and will be referred to as a topic section below. If the sentence includes an index and a value related thereto, the control unit 102 divides the sentence such that the index and value are included in the same topic section. The control unit 102 creates all possible topic section strings. This process will be explained in detail with reference to FIG. 13.

FIG. 13 is a block diagram illustrating the sentence division performed by the trial planning support apparatus 100 of the embodiment of the present invention.

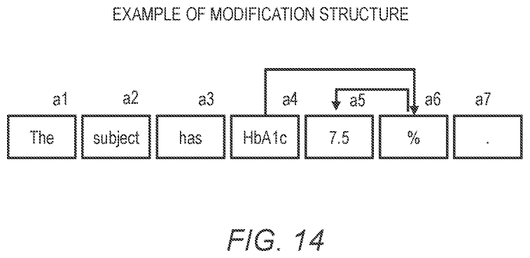

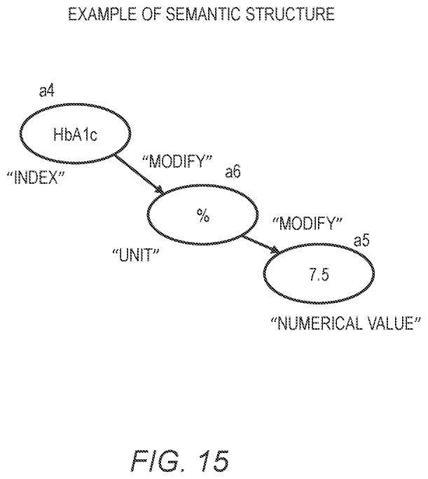

Below, the example of a trial condition sentence made up of words w1 to w10 will be explained. In this example, w2, w4, and w6 are annotated as indices, and w3, w8, and w9 are annotated as values of each index. Also, w2 and w3, w6 and w8, and w6 and w9 each have a modification relationship, or a relationship of an index and a value thereof, in particular. How to determine those relationships will be explained with reference to FIGS. 14 and 15 below.

In Step S1203, the control unit 102 divides such a trial condition sentence into a plurality of text strings each including at least one index, and if there is a value relevant to the index, the index and the value need to be included in the same topic section. The control unit creates all possible topic section strings.

In the example of FIG. 13, since w2, w4, and w6 are each an index, and therefore, a topic border line is drawn between them. However, w2 and w3 have the relationship of the index and value, and thus, the border line of the topic sections (division point) is set between w3 and w4 such that the index and value exist in the same topic section. A topic border line is also set between w4 and w6.

To divide the sentence such that one index is included in a topic section, the sentence can be divided into [P11, P12, p13] and [P21, P22, P23]. P11 and P21 are each a topic section made of w1, w2, and w3. P12 is a topic section made of w4 and w5. P13 is a topic section made of w6 to w10. P22 is a topic section made of w4. P23 is a topic section made of w5 to w10. [P11, P12, P13] and [P21, P22, P23] described above will also be referred to as a topic section string.

As described above, when there are a plurality of division patterns that can divide one sentence such that one topic section always includes at least one index, the control unit creates topic section strings for all division patterns. As a result, in the example of FIG. 13, the following four topic section strings are obtained:
[P11, P12, P13];
[P21, P22, P23];
[P31, P32]; and
[P41, P42].

The control unit 102 stores those strings as a topic section string group in Step S1204.

In Step S1205, the control unit 102 reads out one topic section string from the topic section string group. In step S1206, the control unit 102 calculates the distance between the center of gravity of each sentence cluster created in Step S1106 of FIG. 11 and each topic section of the topic section string. The control unit 102 determines whether there is an unprocessed topic section or not (Step S1207), and if there is, the process returns to Step S1206. This way, the control unit 102 performs the process of S1206 on all of the topic sections in the topic section string.

In Step S1208, the control unit 102 adds up the distances between all of the topic sections in the topic section string and the center of gravity of the cluster, divides the resultant value by the number of topic sections included in the topic section string, thereby calculating the average distance, and obtains the resultant distance as the distance between the topic section string and the center of gravity of the cluster. The control unit 102 determines whether there is an unprocessed topic section string or not (Step S1209), and if there is, the process returns to Step S1205. This way, the control unit 102 performs the calculation of S1208 for all of the topic section strings in the topic section string group.

Lastly, in Step S1210, the control unit 102 finds a topic section string having the smallest distance to the center of gravity of the cluster, which was calculated in Step S1208, employs the sentence division points with which that topic section string was created, and divides the sentence. The control unit 102 then assigns clusters to the divided sections, respectively.

As a result, in Step S1210, it is possible to obtain a topic section string where the topic sections are divided in the best possible way with respect to the existing clusters.

With the process described above, even if one sentence includes a plurality of conditions, the sentence can be divided and each condition is assigned to a cluster, which makes it possible to effectively utilize the past conditions.

It is preferable that, in classifying conditions, the indices characterizing conditions include the same keyword or synonyms, that all values relevant to the indices be identified without limitations, and that the unit be the same.

In order to realize this, when the k-means method is employed for the method to create clusters, for example, if a vector of each sentence is xi, the center of cluster is Vj, the binary index variable is r_ml, and the data point x_m is 1 if included in the first cluster or 0 in all other cases, the optimization algorithm to minimize the distance between the cluster center and data is obtained as in Formula 1.

$$\begin{matrix}Formula 1 & \\ arg {min}_{{v}_{1} - {v}_{k}} {\sum }_{i = 1}^{n} min {r}_{m l} {‖ {x}_{i} - {V}_{j} ‖}^{2} & ( 1 )\end{matrix}$$

Alternatively, another function as in Formula 2 may be used where the word wak relevant to an index appearing in a sentence and the word svk relevant to a value are objective functions, the variation of the parameter attribute is minimized, and the variation of the parameter values is maximized. This way, the distance is calculated such that the distance from the center of gravity of the cluster that includes the same index as that of the sentence is small. As a result, the sentences including different indices are more likely to be classified into different clusters, and the sentences including the same index are more likely to be classified into the same cluster even if the values thereof differ.

$$\begin{matrix}Formula 2 & \\ arg {min}_{{v}_{1} - {v}_{k}} {\sum }_{i = 1}^{n} min {r}_{m l} {‖ {x}_{i} - {V}_{j} ‖}^{2} + \sum {wa}^{2} - \sum {wv}^{2} & ( 2 )\end{matrix}$$

Generally, Formula 1 is used to measure the distance between the topic section and the center of gravity of a cluster, but Formula 2 may alternatively be used for the calculation.

FIG. 14 illustrates an example of the modification structure between words that is referred to by the trial planning support apparatus 100 of the embodiment of the present invention.

The modification structure illustrated in FIG. 14 is an example of the modification structure of the sentence “The subject has HbA1c 7.5%”. In the example of FIG. 4, the clause a4 “HbA1c” modifies the clause a6 “%.” Furthermore, the clause a6 “%” modifies the clause a5 “7.5.” The structural analysis is performed by dividing a text document into clauses, and calculating which clause modifies which clause, for example.

FIG. 15 illustrates an example of the semantic structure that is referred to be the trial planning support apparatus 100 of the embodiment of the present invention.

The semantic analysis is to analyze a text document, and calculate the semantic structure. The semantic structure represents the meaning of a text document by a node indicating the meaning of each word and an arc indicating the semantic relationship between respective nodes. In the example of FIG. 15, information regarding the values of the index such as the disease name, drug name, clinical trial name, or treatment name that appears in the sentences is important for the clinical trial conditions. Thus, a process to recognize a word that indicates an index using a dictionary is performed, for example, and the results thereof are stored as the metadata of each node. Also, the recognition process is performed on values and units, and the results thereof are stored as the metadata for each node. The recognition of indices may be performed by a machine learning method such as CRF.

In the example of FIG. 15, the arc indicates a relationship obtained through the modification analysis.

If values of the index appear in a sentence multiple times, the index to which each value is related needs to be identified. In order to do so, the modification analysis is performed, and if the value is deemed relevant to an index, the process to recognize a value that is to be paired with the index is performed, and if the arc has the relationship of “modify,” the index and the value are deemed relevant to each other.

In the example of FIG. 14, the clauses a1 to a7 correspond to respective nodes. The process to recognize the node a4 “HbA1c″” as an index is performed using a dictionary, for example, and the process to recognize the value node a6 “7.5” and the unit node a5 “%” is performed using the regular expression. Because the node a4 and the node a6, and the node a6 and the node are connected to each other by “modify”, respectively, “7.5%” is recognized as the value of “HbA1c.” This recognition result is stored as the relationship data between index and value illustrated in FIG. 7C.

In the example above, the modification relationship method was described as a process to identify the relationship between index and value, but the relationship may alternatively be identified through machine learning.

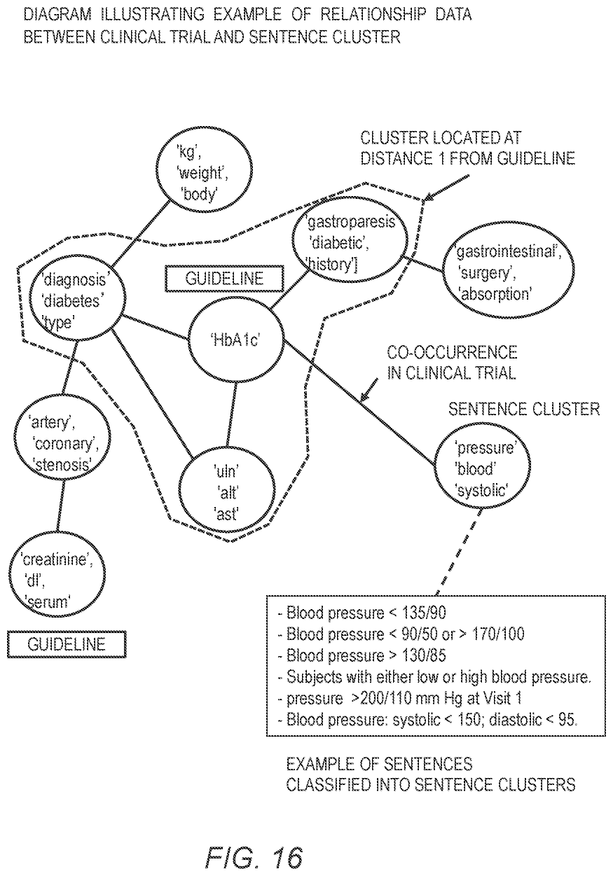

FIG. 16 is a diagram illustrating an example of relationship data between clinical trials and sentence clusters generated by the trial planning support apparatus 100 of the embodiment of the present invention.

The trial condition inter-class relationship analysis unit 118 analyzes the inter-class relationship to find a condition that is always set together with a certain condition. The analysis result is used to help to present relevant conditions based on the relevance in the past cases in creating a protocol.

Therefore, the trial condition inter-class relationship analysis unit 118 includes a co-occurring relationship data creation unit 119 that creates co-occurring relationship data between the clinical trial conditions set in one clinical trial, and a clinical trial condition presentation unit 120 that presents relevant clinical trial conditions in the process of setting clinical trial conditions. The analysis results on the relevance can also be used for data to calculate the presentation order when presenting the classification.

The co-occurring relationship data creation unit 119 of the trial condition inter-class relationship analysis unit 118 totals up the sentence clustering result 127 for each trial, creates binary relationship data of clusters co-occurring in the trial, and stores the data as the relationship data between clinical trials and sentence 129. By connecting clusters based on the binary relationship data of clusters that co-occur in a trial, the cluster map illustrated in FIG. 16 is obtained.

The trial condition sentence such as HbA1c, for example, is specified in the clinical trial guideline, and therefore needs to be included in the trial conditions. Such trial condition sentences need to be flagged so that they are included in the trial conditions as much as possible. Furthermore, it is recommended to include, in the clinical trial conditions, the sentence clusters that have the co-occurring relationship with such condition sentences. In view of this relationship, the sentence clusters represented in FIG. 10 may be displayed such that the cluster of the clinical trial condition sentences with a high degree of necessity is displayed first, and other clusters are displayed in the order of distance of the co-occurring relationship from that cluster. This makes it easier to include the clinical trial condition sentence with a high degree of necessity.

The presentation method described above is merely an example of the method of displaying the relationship of a plurality of clusters including the co-occurring sentences in the documents about the same clinical trial, and the co-occurring relationship may be displayed in other methods.

According to one embodiment of the present invention, it is possible to comprehensively analyze information in designing and evaluating a clinical trial by converging descriptions with very small differences and classifying those descriptions so that clinical trial information is properly extracted and analyzed.

It is also possible to analyze the relationship between the trial conditions and the results based on more comprehensive information.

Furthermore, it is also possible to perform an analysis on the co-occurring relationship between the respective trial conditions, which makes it possible to analyze the relationship between a combination of two or more trial conditions and the trial results.

The present invention is not limited to the embodiment described above, and may include various modification examples. The embodiment described above, for example, was explained in detail such that the present invention is understood more clearly, and shall not necessarily be interpreted as including all of the configurations described above.

Part or all of the respective configurations, functions, processing units, processors, and the like described above may be realized by hardware such as designing with an integrated circuit, for example. The respective configurations, functions, and the like described above may be realized by software with a processor interpreting and executing programs that realize the respective functions. Information such as programs, tables, and files for realizing the respective functions can be stored in a storage device such as a non-volatile semiconductor memory, a hard disk drive, a solid-state drive (SSD), or a computer readable non-temporary data storage medium such as an IC card, SD card, or DVD.

The control lines and information lines needed for explanation were illustrated above, but it does not mean that all of the control lines and information lines in a product were illustrated. In actuality, almost all of the configurations are mutually connected.

What is claimed is:

1. A trial planning support apparatus, comprising:
a processor; and
a storage unit,
wherein the storage unit stores therein data of a plurality of documents about clinical trials implemented in the past, and
wherein the processor is configured to:
receive information about a clinical trial, and search the plurality of documents for a plurality of sentences relevant to the received information;
classify the plurality of sentences that have been found by the search into a plurality of clusters based on a degree of similarity; and
output information about the sentences classified into clusters.

2. The trial planning support apparatus according to claim 1, wherein the processor is configured to:
generate word representation vectors based on the plurality of documents stored in the storage unit such that vectors that represent words that are more likely to co-occur have values closer to each other;
generate sentence representation vectors for the respective plurality of sentences that have been found, based on the word representation vectors that represent respective words included in those sentences; and
classify the plurality of found sentences into a plurality of clusters based on a degree of similarity of the sentence representation vectors.

3. The trial planning support apparatus according to claim 2, wherein the processor is configured to:
classify sentences that are shorter than a prescribed standard, among the plurality of found sentences, into the plurality of clusters based on the sentence representation vectors;
divide a sentence longer than the prescribed standard into a plurality of sections;
generate section representation vectors for the respective plurality of sections, based on word representation vectors that represent words included in those sections; and
classify the plurality of sections into the plurality of clusters based on the section representation vectors.

4. The trial planning support apparatus according to claim 3, wherein the processor is configured to:
divide a sentence longer than the prescribed standard into a plurality of sections such that each section includes at least one index relevant to a clinical trial, and that the index and a value corresponding thereto are included in the same section;
generate section representation vectors that represent the respective plurality of sections for each division pattern if there are a plurality of patterns to divide one sentence, such that each section includes at least said one index, and that the index and a value corresponding thereto are included in the same section;
select a division pattern having the smallest distance between a center of gravity of each of the clusters and the section representation vector that represents each of the sections; and
classify the respective sections into the plurality of clusters based on the distance between a center of gravity of each of the clusters and the section representation vectors of the selected division pattern.

5. The trial planning support apparatus according to claim 4, wherein the processor is configured to:
calculate, if a sentence is shorter than the prescribed standard, a distance between the sentence representation vectors and a center of gravity of each cluster such that a distance between the sentence representation vectors and a center of gravity of a cluster that includes the same index as that of said sentence is small, and assign each sentence to one of the plurality of clusters based on the calculated distance; and
calculate, if a sentence is longer than the prescribed standard, a distance between the section representation vectors and a center of gravity of each cluster such that a distance between the section representation vectors and a center of gravity of a cluster that includes the same index as that of said section is small.

6. The trial planning support apparatus according to claim 4, wherein the index is at least one of a disease name, a clinical trial name, a drug name, an action mechanism name, and a treatment name.

7. The trial planning support apparatus according to claim 1, wherein the processor outputs, for at least one of the clusters, data for displaying sentences classified into the cluster and an index relevant to the clinical trial included in the sentences classified into the cluster, as information about sentences classified into the clusters.

8. The trial planning support apparatus according to claim 7, wherein the processor outputs data for displaying at least one of detailed information of the index included in the sentences classified into the cluster, and a distribution of values corresponding to the index.

9. The trial planning support apparatus according to claim 7, wherein the processor outputs data for displaying a relationship between a plurality of clusters that respectively include sentences that co-occur in documents about the same clinical trial.

10. The trial planning support apparatus according to claim 1, wherein, if the sentences classified into the clusters are sentences about a clinical examination of a drug, the processor outputs data for displaying information that indicates how many of the sentences classified into the clusters are about drugs that went to market.

11. A trial planning support method performed by a computer system including a processor and a storage unit, the storage unit storing therein data of a plurality of documents abouto clinical trials implemented in the past, the method comprising:
a step in which the processor searches the plurality of documents, after receiving information about a clinical trial, for a plurality of sentences relevant to the received information;
a step in which the processor classifies the plurality of sentences that have been found by the search into a plurality of clusters based on a degree of similarity; and
a step in which the processor outputs information about the sentences classified into clusters.

12. A non-transitory computer-readable storage medium that stores a program that control a computer system,
wherein the computer system includes a processor and a storage unit,
wherein the storage unit stores therein data of a plurality of documents about clinical trials implemented in the past,
wherein the program is configured to cause the processor to perform:
a step of searching the plurality of documents for a plurality of sentences relevant to received information after receiving information about a clinical trial;
a step of classifying the plurality of sentences that have been found by the search into a plurality of clusters based on a degree of similarity; and
a step of outputting information about the sentences classified into clusters.
